(19) United States
(12) Patent Application Publication
(10) Pub. No.: US 2026/0069641 A1
(43) Pub. Date: Mar. 12, 2026

(54) GRAM-NEGATIVE BACTERIA FOR USE IN THE PREVENTION AND TREATMENT OF DISEASE IN POULTRY

(71) Applicant: ZIVO BIOSCIENCE, INC., Troy, MI (US)

(72) Inventors: Amy E. Steffek, Royal Oak, MI (US); William P. Pfund, Kalamazoo, MI (US)

(73) Assignee: ZIVO BIOSCIENCE, INC., Troy, MI (US)

(21) Appl. No.: 19/238,384

(22) Filed: Jun. 14, 2025

Related U.S. Application Data
(60) Provisional application No. 63/660,242, filed on Jun. 14, 2024.

(51) Int. Cl. A61K 35/74; A23K 10/16; A23K 20/163; A23K 50/75; A61K 31/739; A61K 39/00; A61K 39/12; A61K 39/145; A61P 37/04

(52) U.S. Cl. CPC A61K 35/74; A23K 10/16; A23K 20/163; A23K 50/75; A61K 31/739; A61K 39/12; A61K 39/145; A61P 37/04; A61K 2039/552

(57) ABSTRACT

A bacteria-based compound for the prevention and treatment of a number of strains of avian influenza via oral intake by way of feed or drinking water or both. The agent is an inventive treatment compound comprising a bacterial-based culture which is directed to the prevention and treatment of avian influenza primarily for use in commercial birds such as poultry. The inventive treatment compound disclosed herein is produced from a lipopolysaccharide of a Gram-negative bacterial strain of the Brevundimonas group. The compound may be delivered to poultry by way of liquid or dry feed according to an effective prescribed regimen. A similar treatment approach may be taken as well after the bird is infected. Use of the composition is effective in the prevention and treatment of a variety of avian influenza, including high pathogenicity avian influenza, low pathogenicity avian influenza, Newcastle disease virus, infectious bronchitis disease virus, and infectious laryngotracheitis virus.

### CROSS REFERENCE TO RELATED APPLICATION

This application is a US. Non-provisional patent application of U.S. Provisional Patent Application No. 63/660,242, entitled “Gram-Negative Bacteria for Use in the Prevention and Treatment of Avian Influenza,” filed Jun. 14, 2024, which is herein incorporated by reference in its entirety for all purposes.

### TECHNICAL FIELD

The present invention relates to the use of a bacteria-based compound for the prevention and treatment of avian influenza via oral intake by way of feed or drinking water or both. More particularly, the present invention relates to a compound and the use of a compound such as that derived from an active compound of Gram-negative bacterial strains that are members of the genus Brevundimonas. The aforementioned compound selectively modulates Toll-like receptors (TLRs) for the prevention and treatment of viral diseases such as avian influenza via a direct effect on innate and adaptive immune pathways.

### BACKGROUND OF THE INVENTION

The overall health of the respiratory system of all animals, and specifically in poultry, is of critical concern in many industries. Poor respiratory health results in lowered production rates and increased morality in penned animals. Positive respiratory health is directly related to the overall performance, health and welfare of animals used in the food production industry. Respiratory health in birds is itself highly complex, depending as it does on a variety of factors, including nutrition and immunology. The good health of the gastrointestinal tract is also critical to the overall good health of poultry.

The primary causative agents for compromised respiratory systems in birds is avian influenza. Avian influenza is a viral infection which may be found in a broad range of avian species around the world having been reported in many countries. Viruses of the family Orthomyxoviridae of the genus Alphainfluenzavirus cause the viral infection. the basis for avian influenza. The disease may impact domestic poultry while asymptomatic carriers of the infection frequently include wild waterfowl and shorebirds. It is possible for avian influenza to occur in humans although such occurrences are believed to be rare. Virus isolation is the technique commonly used for confirmation of the disease. To varying degrees, the health of the health of the bird may also be compromised by avian influenza.

The different strains of avian influenza are capable of infecting poultry, resulting in relatively mild symptoms in some instances or, at the other extreme, death brought on only a brief time after infection. Many of the causative viruses are easily transmitted in the air, thus making prevention very difficult, particularly in commercial poultry settings where the birds are often kept in close quarters. While only rarely found in humans, avian influenza may nonetheless jump species as an occupation hazard to those in various aspects of the poultry industry. Possible human infections may be known to those working in abattoirs or as vaccinators in which case known methods of treatment and prevention by way of one or more vaccines poses a health problem. Personnel working in laboratories may also be at risk.

Some birds infected with avian influenza may be asymptomatic while some demonstrate only mild symptoms. Conversely, infection in some birds may be hyperacute and fatal. The various strains of avian influenza impact birds in different ways. A variety of strains of avian influenza are known including high pathogenicity avian influenza, low pathogenicity avian influenza, Newcastle disease virus, infectious bronchitis disease virus, and infectious laryngotracheitis virus. Other strains of the virus are known and, due to occasional mutations, add continuously to the complex patchwork of known avian influenzas.

High pathogenicity avian influenza (HPAI), sometimes called “fowl plague,” is perhaps the most serious of avian influenza strains given its highly contagious and often fatal results in poultry. It is possible in many cases for mortality in a flock to be as high as 100%. Infected peracute birds often display few or no signs or symptoms of illness prior to death. Conversely, in acute cases signs may include edema of the head or comb as well as in the feet. Surviving peracute cases often suffer CNS involvement characterized in paralysis and drooping wings.

Low pathogenicity avian influenza (LPAI) is the most common strain of avian influenza. Birds infected with LPAI present clinical manifestations such as coughing and both nasal and ocular discharge. Swollen sinuses are also common in LPAI-infected birds. Post-mortem lesions in the respiratory tract are usually associated with the lungs and trachea. Milder than HPAI, infected birds may demonstrate reduced decreased egg production or, in more acute cases, may suffer renal failure.

Newcastle disease virus (NDV) sometimes alternatively known as velogenic viscerotropic disease (VVND) or Asiatic Newcastle disease (AND) is commonly diagnosed in domestic fowl as a chronic ailment. NDV has a particularly high mortality rate in poultry of between 50% and 80%. However, the VVND strain is perhaps the most lethal of the strain of the Newcastle diseases presenting an acute and fatal infection of a variety of avian species of all ages. The VVND strain results in hemorrhagic lesions of the gastrointestinal tract. The VVND strain is highly resistant to known treatment methods. The Newcastle family of viral diseases is highly transmissible by wind and thus is difficult to control.

Infectious bronchitis disease (IBD) virus is an acute and highly contagious viral infection of birds and is commonly found in poultry. Infected birds suffer from a variety of respiratory signs and demonstrate renal failure. The transmission pattern of the virus is typically by wind, a situation which creates particular challenges for keeping poultry because of its potential for spreading rapidly within the flock. Increasing the challenges associated with IBD are the hosts themselves which become carriers and shedders of the virus for weeks or months after initial infection.

Infectious laryngotracheitis virus (ILTV) is a disease commonly found in poultry. The disease is typically characterized by respiratory difficulties including coughing which results in bloody exudate. Acutely infected poultry most commonly pass the disease to non-infected birds by air transmission. Birds which successfully recover from ILTV may shed the virus for an extended time. The disease frequently allows infected birds to recover typically within a matter of a couple of weeks.

Limited prevention and treatment measures for responding to avian influenza are known but are presented with challenges. Practical measures involved in prevention are available and primarily include exclusion strategies given the relatively high level of contagion within a flock. Vaccines which are antigenically matched may also provide a suitable treatment provided administration is undertaken according to appropriate protocols. Vaccines prepared from specific influenza of the same hemagglutinin subtype have perhaps the greatest chance of overall success although some require approved of state veterinary services.

Once infected with a strain of avian influenza, flocks are often culled to minimize risk of further infections particularly in the case of HPAI viruses. Treatment for the other forms of avian influenza, such as LPAI, may be less dramatic. Symptomatic and supportive assistance to infected birds can be the most common treatment strategies. Perhaps the greatest challenge to the use of vaccines in the treatment of avian influenza is that the appropriate vaccines are typically strain or serotype specific. Accordingly, new vaccines will need to be continuously developed in response to mutations of existing strains or the arrival of entirely new strains.

It is thus desirable to develop an agent and method of treatment for avian influenza in animals, particularly in poultry, thereby strengthening overall health in the animals, and particularly in the animal's respiratory and gastrointestinal systems.

### SUMMARY OF THE INVENTION

The disclosed inventive composition provides an improved agent and treatment method for a number of strains of avian influenza. The agent is an inventive treatment compound comprising a bacterial-based culture which is directed to the prevention and treatment of avian influenza primarily for use in commercial birds such as poultry but may also find beneficial use in humans. The compound is easy to administer and is cost effective.

The inventive treatment compound disclosed herein is produced from a lipopolysaccharide (LPS) of a Gram-negative bacterial strain that is a member of the Brevundimonas group, a relatively rare bacterium. Particularly, the preferred bacterium incorporated into the present inventive compound is Brevundimonas nasdae.

As preventive measure, the disclosed treatment compound may be delivered to poultry by way of liquid or dry feed according to an effective prescribed regimen. A similar treatment approach may be taken as well after the bird is infected.

Accordingly, consumption of the inventive treatment compound by way of liquid or dry feed produces a broad range of health benefits and has proven effective in the prevention and treatment of a wide variety of avian influenza, including but not limited to high pathogenicity avian influenza, low pathogenicity avian influenza, Newcastle disease virus, infectious bronchitis disease virus, and infectious laryngotracheitis virus. The disclosed treatment compound may also prove to both prevent and treat mutated forms of avian influenza.

Among the many health benefits resulting from consumption of the inventive treatment compound described herein is efficacy to both high and low pathogenicity avian influenza (HPAI and LPAI) as well as Newcastle Disease Virus (NDV), Infectious Bronchitis Disease Virus (IBD) and Infectious Laryngotracheitis Virus (ILTV), all discussed above.

The compound of the disclosed inventive concept is combined with conventional feed for administration to animals, such as poultry. During the treatment period, the disclosed compound derived from a lipopolysaccharide (LPS) of gram-negative bacteria is administered to the animal by way of poultry feed, drinking water, or both. The composition itself is a natural product and thus has no adverse environmental impact unlike known antibiotic regimens. Thus, the approach of the disclosed inventive concept stands in sharp contrast to known and commonly used disease treatments.

### DESCRIPTION OF THE DRAWINGS

For a more complete understanding of this invention, reference should now be made to the accompanying figures in which:

FIG. 1 is a graph illustrating average pen body weight at Day 1 according to the first study;

FIG. 2 is a graph illustrating enhanced feed conversion ratio in cocci-challenged subject animals fed a composition according to the disclosed inventive concept according to a first study;

FIG. 3 is a graph illustrating feed intake comparison in cocci-challenged subject animals fed a composition according to the disclosed inventive concept according to the first study;

FIG. 4 is a graph illustrating average pen body weight comparison in cocci-challenged subject animals fed a composition according to the disclosed inventive concept according to the first study;

FIG. 5 is a graph illustrating reduction of mortality due to Eimeria infection in cocci-challenged subject animals fed a composition according to the disclosed inventive concept according to the first study;

FIG. 6 is a graph illustrating reduction of an intestinal lesion score of the duodenum in cocci-challenged subject animals fed a composition according to the disclosed inventive concept according to the first study;

FIG. 7 is a graph illustrating reduction of an intestinal lesion score of the ileum in cocci-challenged subject animals fed a composition according to the disclosed inventive concept according to the first study;

FIG. 8 is a graph illustrating reduction of an intestinal lesion score of the ceca in cocci-challenged subject animals fed a composition according to the disclosed inventive concept according to the first study;

FIG. 9 is a graph illustrating restoration/protection of ileum villi cell height in cocci-challenged subject animals fed a composition according to the disclosed inventive concept according to the first study;

FIG. 10 is a graph illustrating restoration/protection of ileum crypt depth in cocci-challenged subject animals fed a composition according to the disclosed inventive concept according to the first study;

FIG. 11 is a graph illustrating restoration/protection of ileum villi height to crypt depth ratios in cocci-challenged subject animals fed a composition according to the disclosed inventive concept according to the first study;

FIG. 12 is a graph illustrating reduction of Eimeria in the duodenum gut in cocci-challenged subject animals fed a composition according to the disclosed inventive concept according to the first study;

FIG. 13 is a graph illustrating reduction of Eimeria in the ileum in cocci-challenged subject animals fed a composition according to the disclosed inventive concept according to the first study;

FIG. 14 is a graph illustrating reduction of Eimeria in the ceca in cocci-challenged subject animals fed a composition according to the disclosed inventive concept according to the first study;

FIG. 15 is a graph illustrating the groups, the test material administered, the inclusion amounts, the group to which the material was fed, the status of coccidiosis challenge, and the lesion scoring dates for the second study;

FIG. 16 is a graph illustrating average pen body weight at Day 1 according to the second study;

FIG. 17 is a graph illustrating feed intake comparison in cocci-challenged subject animals fed a composition according to the disclosed inventive concept according to the second study at Day 28;

FIG. 18 is a graph illustrating enhanced feed conversion ratio in cocci-challenged subject animals fed a composition according to the disclosed inventive concept according to a second study at Days 1-28;

FIG. 19 is a graph illustrating reduction of mortality due to Eimeria infection in cocci-challenged subject animals fed a composition according to the disclosed inventive concept according to the second study at Days 1-28;

FIG. 20 is a graph illustrating average pen body weight gain comparison in cocci-challenged subject animals fed a composition according to the disclosed inventive concept according to the second study;

FIG. 21 is a graph illustrating reduction of an intestinal lesion score of the duodenum in cocci-challenged subject animals fed a composition according to the disclosed inventive concept according to the second study at Day 28;

FIG. 22 is a graph illustrating the coccidia incidence score in the small intestine in cocci-challenged subject animals fed a composition according to the disclosed inventive concept according to the second study at Day 28;

FIG. 23 is a graph illustrating results from Applicants' first Low Pathogenicity Avian Influenza (LPAI) study which provide evidence that the disclosed composition mitigates the spread of H7N2 virus among poultry; and

FIG. 24 is a graph illustrating results from Applicants' second Low Pathogenicity Avian Influenza (LPAI) study which provide further evidence that the disclosed composition mitigates the spread of H7N2 virus among poultry.

The reference “Significance (P<0.05)” in the figures refers to means within a row without a common superscript are significantly different (P<0.05), as determined by Duncan's New Multiple Range Test (MRT 1955).

### DETAILED DESCRIPTION OF THE PREFERRED EMBODIMENT

In the following description, various operating parameters and components are described for different constructed embodiments. These specific parameters and components are included as examples and are not meant to be limiting. Unless otherwise noted, all technical and scientific terms used herein are to be accorded their common meanings as would be understood by one having ordinary skill in the art.

Applicants' kinomics data generated by studies involving algal biomass containing Brevundimonas show systemic enhancement and maturation of a host of immune pathways. Based on past studies which show that Brevundimonas biomass has already been shown to exhibit broad spectrum efficacy for certain parasitic diseases (e.g., coccidiosis caused by Eimeria) as well as a reduction of parasitic bacteria in the chicken gastrointestinal tract, it is clear that efficacy against viral disease is a reasonable expectation, such efficacy having been verified by Applicants' data. In addition, the Brevundimonas biomass has been proven to exhibit good TLR agonist activity. Microbial ligands or TLR ligands have been shown to act as prophylactic agents for protection against avian influenza infection in poultry. Furthermore, Applicants' studies suggest that TLR ligands are effective in reducing avian influenza shedding after experimental challenge.

The inventive composition and method of treatment developed by Applicants offer several advantages over commonly known treatment methods and compositions. These include but are not limited to the circumstance that vaccines are typically strain or serotype specific. As a result, new vaccines will continually be required as the virus mutates or as new strains appear. In contrast, the disclosed inventive treatment composition and method enhances the immune response regardless of the virus, strain, or serotype, avoiding the limited lives of known treatment approaches. Furthermore, the disclosed inventive treatment method and composition is effective both as a stand-alone product and as an adjuvant when co-administered with a vaccine, such as those vaccines proven for use in the prevention of coccidiosis.

In addition, the inventive composition and method of treatment developed by Applicants has shown the potential of delivering TLR agonists in the form of whole bacterial biomass may enhance the duration of their effect. Limitations of the use of TLR ligands in clinical application is their short half-life and expeditious clearance from the body. With these limitations in mind, a range of methods (e.g., encapsulation or adsorption) have been used to increase product half-life. The inventive composition disclosed herein accomplishes the same effect without the need for such methods, thereby increasing the practical and easy use of the treatment composition while reducing costs.

Representing additional advantages over known methods of disease treatment, the inventive composition and method of treatment developed by Applicants is delivered to the bird by way of oral delivery in feed, an uncommon method for treating avian influenza. Accordingly, the delivery method of the present invention is extremely easy to adopt in the broiler or egg production environment.

The biomass of the inventive composition and method of treatment disclosed herein delivers multiple actives in combination. While TLR2 and TLR4 agonists have been shown to be effective against avian influenza, these agonists are conventionally delivered and studied separately. Conversely, the biomass of the present invention delivers these actives (and likely others) in combination to allow synergistic effects to increase efficacy.

### The Compound Used in Treatment

The disclosed method of treatment preferably, but not absolutely, utilizes a compound generally derived from an active compound that may be found in the cell wall of a Gram-negative bacterial strain that is a member of the genus Brevundimonas.

As used herein, “modulator” refers to an activator, an inhibitor, or both. Modulation may be the result of activity by at least one Toll-like receptor (TLR), such as TLR4 or possibly TLR2 as well as other Toll-like receptors. As used herein, the term “inhibitor” refers to a molecule that reduces or attenuates the activity induced by another molecule. By way of example, a compound that might block the LPS-dependent activation of TLR4 present on the surface of immune cells in humans and animals would be regarded as an inhibitor of this particular pathway.

As used herein, the term “culture” is defined as microorganisms (either isolated or in combination) that grow in a liquid medium. Unless expressly stated otherwise, the term “biomass” refers to the microorganism cells (with the liquid culture medium removed). The “biomass” can be wet material or dried material. Unless expressly stated otherwise, the term “supernatant” is defined as the culture medium in which the biomass is grown that contains excreted compounds from the biomass. Supernatant is obtained by growing biomass in culture medium for an appropriate length of time and then removing the microorganism cells by filtration and/or centrifugation.

Brevundimonas is a Gram-negative aerobic, non-fermenting bacterium that can grow under a variety of conditions. It is capable of metabolically utilizing several natural compounds generated by plants or algae. Embodiments of the compound used in the treatment of disease as set forth herein include one or more LPS/Lipid A compounds produced by Gram-negative bacterial strains for use as selective modulators of the TLR signaling pathway, such as the TLR4 pathway. The disclosed inventive composition involves any combination of three fundamental steps: (1) the Gram-negative bacteria produces LPS/Lipid A compounds; (2) the LPS/Lipid compounds modulate TLR4 activity through activation or inhibition; and (3) a downstream effect results in enhanced innate and adaptive immune processes, thereby aiding in the treatment of coccidiosis, necrotic enteritis, and other conditions related to gut inflammation.

In an embodiment, the LPS/Lipid A compounds used as selective modulators of the TLR4 signaling pathway are produced from a strain of the genus Brevundimonas. The Brevundimonas strain may be a naturally occurring strain found in an algal biomass. Accordingly, embodiments of the compound used in the treatment of disease according to the present disclosure are directed to one or more LPS/Lipid A compounds produced by a Gram-negative bacterial strain of the group Brevundimonas for use as selective modulators of the TLR signaling pathway.

The LPS/Lipid A compound employed herein may preferably be obtained from a member of the group comprising but not necessarily limited to any one of Brevundimonas vesicularis, Brevundimonas nasdae, Brevundimonas intermedia, Brevundimonas aurantiaca, Brevundimonas mediterranea, Brevundimonas albigilva, and Brevundimonas huaxiensis by any suitable method. In some examples, multiple types of LPS extraction protocols are employed to obtain an LPS compound from the bacteria, and extraction procedures may be performed more than once. Once the LPS compound is extracted and purified from the bacteria, the Lipid A fraction may be prepared by acid hydrolysis or other suitable techniques. In some examples, analysis of the structure of the LPS compound is performed using routine methods in the art, including using mass spectrometry, gas chromatography, or both.

The one or more LPS/Lipid A compounds derived from Gram-negative bacterial strains of the genus Brevundimonas, may selectively modulate the TLR4 signaling pathway to alter inflammatory responses and to improve immune health in a variety of uses and applications. In an embodiment, the LPS/Lipid A compound derived from species of the genus Brevundimonas may be incorporated within a feed ingredient to improve gut health of poultry.

The disclosed LPS/Lipid A compound derived from a species of the genus Brevundimonas may be used to improve the health of poultry or other animals through a variety of mechanisms. For example, the LPS/Lipid A compound may protect against internal inflammation in poultry by negatively regulating inflammatory mediators via the downregulation of TLR4 expression and the downstream inhibition of NF-kappa B activation in a typical inflammatory cascade. In another example, the LPS/Lipid A compound may inhibit the activation of TLR4 in poultry by interfering with cysteine residue-mediated receptor dimerization. In yet another example, the LPS/Lipid A compound may inhibit the ability of non-infectious and infectious stimuli to interact with TLR4 and trigger a pro-inflammatory response, thereby improving poultry gut integrity. In a further example, the LPS/Lipid A compound may modulate TLR4 through either ligand-dependent or ligand-independent activation. As another example, the LPS/Lipid A compound may act in concert with other TLR agonists to provide a heightened immune response, while reducing the metabolic costs to the host.

The treatment compound is dried biomass containing Gram-negative bacteria of the genus Brevundimonas, or compounds derived therefrom, provided in drinking water or as animal feed. In animal feed, once the biomass and feed additive are combined to the preferred premix level, the combined batch is poured or administered evenly into a ribbon mixer containing finished feed. The combined batch is preferably provided in an amount of between about 100.0 g dried Brevundimonas per ton of finished feed and about 150.0 g per ton of finished feed, is more preferably provided in an amount of between about 120.0 g per ton of finished feed and 130.0 g per ton of finished feed and is most preferably though not exclusively provided in an amount of about 125.0 g dried Brevundimonas per ton of feed with good efficacy without being wasteful.

As an alternative, the combined batch may be provided in an amount of between about 100.0 g dried Brevundimonas per ton of finished feed and about 300.0 g per ton of finished feed, is more preferably provided in an amount of between about 150.0 g per ton of finished feed and 250.0 g per ton of finished feed, and is most preferably though not exclusively provided as an alternative in an amount of about 200.0 g dried Brevundimonas per ton of feed with good efficacy without being wasteful.

As an active fraction or isolated molecule, the amounts administered are less than the amounts needed for whole cells in that the amount required for disease prevention and treatment depend on the potency of the material. For example, where purified LPS is used in feed the ratio is between about 0.1 g per ton of finished feed and about 3.0 g per ton of finished feed with the preferred range being between about 0.16 g per ton of finished feed and about 2.5 g per ton of finished feed.

Where the active is administered in water, where a supernatant is the source from an algal culture, the amount is between about 0.1 and 0.3 mL per liter of drinking water with 0.2 mL per liter being most preferred. Where a semi-pure LPS fraction is utilized, the amount is between about 1.0 and 2.0 mg/L with the preferred amount being 1.5 mg/L. Where a purified LPS is utilized, the amount is between about 0.001-0.03 mg/L with between about 0.002-0.02 g/L being most preferred.

The composition of the present invention demonstrates a variety of benefits even beyond those set forth above. A significant finding was that administration of the composition in combination with a vaccine resulted in a delay of transmission of avian influenza from infected birds to heathy birds, thus providing additional time for the development of immunity in healthy birds.

The composition as described was provided to the bird at the same time one or more vaccines selected from known vaccines, including Arepanrix® (D Biomedical Corporation of Quebec) and Audenz® (Seqirus, Inc.) was administered. The selected vaccine was dosed in a single dose per chick via a coarse spray vaccination. The vaccine suspension was diluted with the appropriate solvent and water whereupon a 10,000-dose vial was diluted in 2.5 liters of water for a spray cabinet application.

Related to this finding was the discovery that use of the treatment composition increased protection in the time between vaccination and the development of immunity. In addition to these advantages, treatment using the present composition also demonstrated an increased level of immunity when used in conjunction with a vaccine. The disclosed composition and treatment method also provides relief for other diseases affecting poultry, including but not limited to Infectious Bursal Disease (IBD), also known as Gumboro Disease. A highly contagious viral disease, IBD affects young chickens, turkeys, and ducks between about three and six weeks old primarily by giving rise to immunosuppression and, ultimately, early mortality.

### Investigations

Investigations were undertaken to determine the response and efficacy of a feed ingredients containing Brevundimonas biomass incorporated at a specific amount into a commercial-type corn-soybean diet and fed to floor-pen raised broilers challenged with Eimeria to cause coccidiosis. The investigations were undertaken over a 28-42 day period, from Day 0 to Day 28 or Day 0 to Day 42. Twenty investigations were performed under a wide range of test conditions and configurations designed to represent the broad range of conditions expected in commercial poultry houses. Several of the investigations were performed by administering biomass from the isolated active Brevundimonas bacterial strain. All other investigations involved administering culture biomass containing the active bacterial strain as the test material.

Investigations were conducted in both battery cages and floor pens, using both mash and pelleted feed rations using appropriate numbers of birds and replicates to validate statistically significant findings (P<0.05). In most investigations, a disease challenge involving three species of Eimeria (E. tenella, E. maxima, and E. acervulina) was applied, but the timing of the challenge was varied to validate efficacy under different infection scenarios. Investigation endpoints included performance parameters (e.g., feed intake, body weight gain, FCR), disease endpoints (e.g., mortality, lesion score, intestinal morphology, parasite enumeration).

While a majority of investigations were conducted over the full 42-day broiler grow-out period, several investigations were truncated to 28-days in order to streamline the product development cycle after validating that the results from 28-day investigations are fully representative and predictive of results for full-length investigations. Two specific studies selected from the full research program are discussed here.

### First Study

Chicks were obtained within twelve hours of hatching from fecal contaminated flocks at a commercial hatchery on Day 0 (hatch and placement day). Three-hundred mixed-sex broiler chicks (50:50 sex ratio) were randomly assigned on Day 0 by individual weights to each of several test group pens, each with replicates. Only antibiotic-free birds were sourced, and no coccidiosis vaccine was administered at the hatchery or at any time during the study. Chicks were evaluated upon receipt for signs of disease or other complications that could affect study outcome. Weak birds were humanely sacrificed. Birds were not replaced during the study.

Following examination, chicks were weighed and allocated to pens for the various treatment groups using a randomized block design. Weight distribution across the treatment groups was assessed prior to feeding by comparing the individual test groups' standard deviations of the mean against that of the control group. Weight distribution across the groups was considered acceptable for this study when differences between control and test groups were within one standard deviation.

All birds received nutritionally adequate diets as pellets (crumbled pellets for Days 1-14). Birds were fed their respective treatment diets ad libitum from day of hatch to 28 days of age. (The typical average market age is 42 days.) Birds were raised on built-up litter to further mimic stress conditions typically experienced in poultry production.

All diets were offered ad libitum without restrictions to full-fed consumption, except for an 8-hour fasting period prior to cocci-challenge on Day 7 when all birds assigned to challenge test groups received oocyst-inoculated feed containing a mixture of Eimeria acervulina, Eimeria maxima, and Eimeria tenella. Dietary requirements for protein, lysine, methionine, methionine+cystine, arginine, threonine, tryptophan, total phosphorus, available phosphorus, total calcium, dietary sodium, and dietary choline were met by adjusting the concentrations of corn and soybean meal ingredients, as well as other minor ingredients commonly used in poultry production.

Throughout the study, birds were observed at least three times daily for overall health, behavior, and evidence of toxicity. Pens were monitored for environmental conditions, including temperature, lighting, water, feed, litter condition, and unanticipated house conditions/events. Pens were checked daily for mortality. Examinations were performed on all broilers found dead or moribund. Mortalities were recorded (date and weight) and examined (both internal and external body mass).

Cocci-Challenge—On Day 7, all birds assigned to challenged test groups received oocyst-inoculated feed containing a mixture of Eimeria acervulina, Eimeria maxima, and Eimeria tenella. Adequate feed was precisely weighed and provided to birds to consume at the rate of 100% fill-capacity on average. Prior to the challenge, all birds were starved for eight hours. Inoculated feed was provided to the birds. Following a specific time, all remaining inoculated feed was removed and weighed to assure equal consumption per pen and per bird. The quantity of feed (both placed and withdrawn) was recorded on each pen's feed record.

### First Study Evaluation

Differences between groups were evaluated at P<0.05, employing Treatment×Replicate RCB (Randomized Complete Block) design. Particularly and as noted, “Significance (P<0.05)” refers to means for groups not sharing common letter designations in the figures which indicates significant difference (P<0.05) between said groups, as determined by Duncan's New Multiple Range Test (MRT 1955).

The relevant evaluation results from the first study are set forth in the following chart and are referenced below by individual graphically represented figures:

| | A | B | C | D | E | F | G |
|---|---|---|---|---|---|---|---|
| Means | | | | | | | |
| 1. No Tx, | 1.527 | 1.852 | 1418.47 | 88.502 | 0.950 | 0.067 | 0.075 |
| No | | | | | | | |
| Challenge | | | | | | | |
| 2. No Tx, | 1.595 | 17.593 | 1365.56 | 98.530 | 2.483 | 2.525 | 2.492 |
| Cocci | | | | | | | |
| 3. Coban, | 1.527 | 1.852 | 1430.88 | 89.499 | 0.525 | 0.433 | 0.583 |
| Cocci | | | | | | | |
| 4. Brev, | 1.536 | 3.241 | 1446.26 | 91.538 | 0.450 | 0.500 | 0.483 |
| Cocci | | | | | | | |
| Significance | | | | | | | |
| (P < 0.05) | | | | | | | |
| 1. No Tx, | a | a | a | a | b | a | a |
| No | | | | | | | |
| Challenge | | | | | | | |
| 2. No Tx, | b | b | b | b | c | c | c |
| Cocci | | | | | | | |
| 3. Coban, | a | a | a | a | ab | b | b |
| Cocci | | | | | | | |
| 4. Brev, | a | a | a | a | a | b | b |
| Cocci | | | | | | | |

KEY:
A. Feed Conversion Ratio Corrected Days 1-28
B. % Mortality Days 1-28
C. Avg Pen Body Weight (g) Day 28
D. Feed Intake (g/bird/day) Day 28
E. Lesion Score Duodenum Day 28
F. Lesion Score Ileum Day 28
G. Lesion Score Ceca Day 28

### Benefits of Disclosed Inventive Composition in Animal Growth Performance

The disclosed inventive composition offers several benefits not seen in known feed compositions. The results from the 20 studies performed demonstrate that the actives of the disclosed inventive composition consistently improve the efficiency with which broiler chickens convert feed into body mass gains in the presence of a disease challenge. More specifically as illustrated in the representative study described above in conjunction with the attached figures, Eimeria-challenged birds ingesting the actives of the disclosed inventive composition consistently show statistically significant improvements in FCR compared to non-treated, cocci-challenged birds. In addition to deliberate Eimeria inoculation early in life, studies were conducted on built-up litter providing ongoing exposure to Eimeria and other environmental pathogens during the course of the studies. Despite this, the FCR status of birds fed the disclosed inventive composition was consistently found to be not statistically different from unchallenged birds and challenged birds treated with leading ionophore products commonly used in poultry production, such as Salinomycin, Coban® (Monensin, USP), Elanco Animal Health, and Maxiban® (Narasin and nicarbazin), Elanco Animal Health. These improvements in FCR are reliably shown to be due to increases in body weight and not due to increased feed intake.

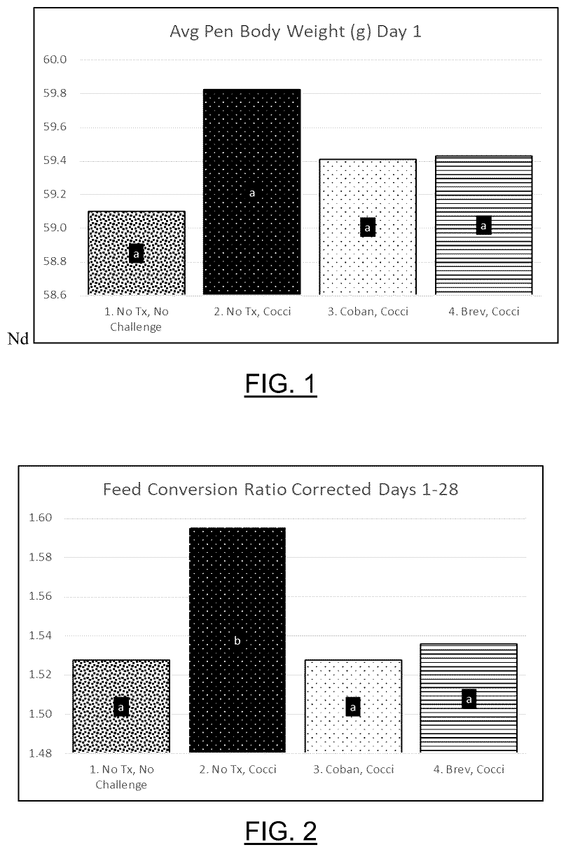

Day 1 Body Weight Evaluation—Referring to FIG. 1, Average Body Weight at Day 1 is illustrated. Body weights are not statistically different among groups at this stage, ranging from a high weight of 59.096 g for birds assigned to the unchallenged and untreated groups to 59.821 g for birds assigned to the challenged and untreated groups:

| | Avg Pen |
|---|---|
| | Body Weight |
| Means | (g) Day 1 |
| 1. No Tx, No Challenge | 59.096 |
| 2. No Tx, Cocci | 59.821 |
| 3. Coban, Cocci | 59.407 |
| 4. Brev, Cocci | 59.429 |
| Significance (P < 0.05) | |
| 1. No Tx, No Challenge | a |
| 2. No Tx, Cocci | a |
| 3. Coban, Cocci | a |
| 4. Brev, Cocci | a |

Feed Conversion Rate—As set forth in FIG. 2 which is a graph illustrating enhanced feed conversion ratio in cocci-challenged subject animals fed a composition according to the disclosed inventive concept, mortality-corrected FCR was determined from day of hatch through Day 28 for control birds receiving no Eimeria challenge and no treatment (first bar) and birds challenged with Eimeria on Day 7 but untreated (second bar), challenged and treated with Coban® (third bar), or challenged and treated with the immune modulator of the disclosed composition (fourth bar). Growth performance as measured by FCR for the group fed the disclosed inventive composition is dramatically improved compared to the challenged/untreated group and not statistically different from the unchallenged/untreated control group or the challenged/Coban® treated group.

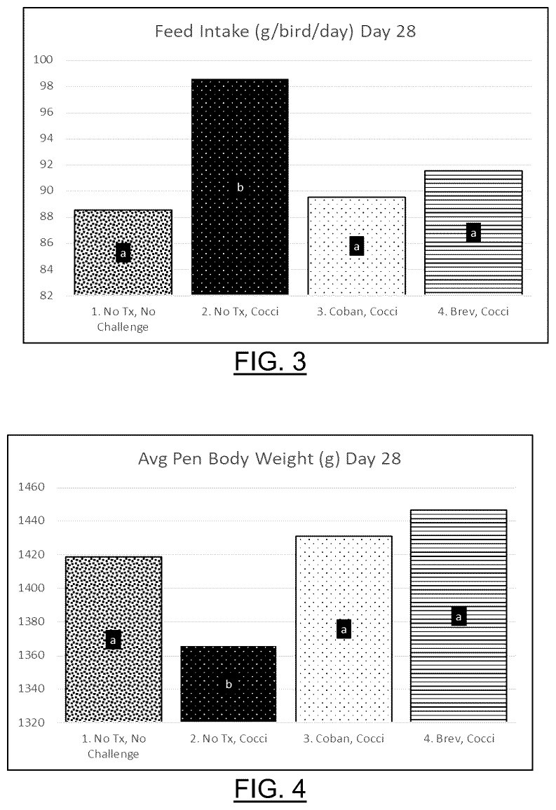

Feed Intake—As set forth in FIG. 3 which is a graph illustrating feed intake comparison in subject animals fed a composition according to the disclosed inventive concept, feed intake was measured from day of hatch through Day 28 for control birds receiving no Eimeria challenge and no treatment (first bar) and birds challenged with Eimeria on Day 7 but untreated (second bar), challenged and treated with Coban® (third bar), or challenged and treated with the immune modulator of the disclosed composition (fourth bar). As expected, feed intake increased significantly in challenged/untreated birds. In contrast, feed intake of challenged birds receiving the immune modulating actives of the disclosed composition was not significantly different than that of the unchallenged/untreated control group or the challenged/Coban® treated group.

Body Weight—As set forth in FIG. 4 which is a graph illustrating average pen body weight comparison in subject animals fed a composition according to the disclosed inventive concept, average pen body weight was measured from day of hatch through Day 28 for control birds receiving no Eimeria challenge and no treatment (first bar) and birds challenged with Eimeria on Day 7 but untreated (second bar), challenged and treated with Coban® (third bar), or challenged and treated with the immune modulator of the disclosed composition (fourth bar). As expected, body weight was significantly decreased in challenged/untreated birds compared to the other groups. In contrast, body weight of challenged birds receiving the immune modulating actives of the disclosed composition was not significantly different than that of the unchallenged/untreated control group or the challenged/Coban® treated group.

Mortality—Across all 20 studies, actives of the disclosed composition consistently reduced mortality in Eimeria-challenged broiler chickens, resulting in mortality levels not statistically different from those observed in unchallenged control groups and challenged birds treated with leading ionophore products commonly used in poultry production.

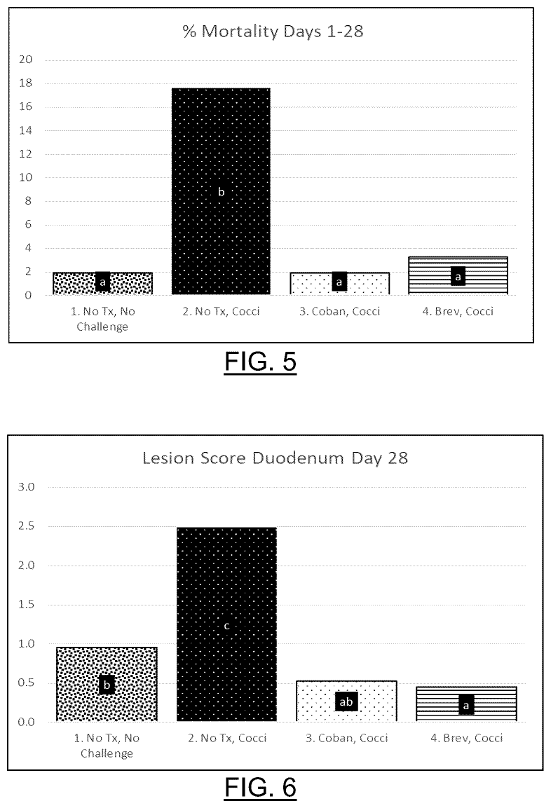

As set forth in FIG. 5 which is a graph illustrating reduction of mortality due to Eimeria infection in subject animals fed a composition according to the disclosed inventive concept, the percent mortality was calculated from day of hatch through Day 28 for control birds receiving no Eimeria challenge and no treatment (first bar) and birds challenged with Eimeria on Day 7 but untreated (second bar), challenged and treated with Coban® (third bar), or challenged and treated with the immune modulator of the disclosed composition (fourth bar). Mortality in the challenged/untreated group was approximately 17% indicating that the Eimeria challenge resulted in significant disease. In contrast, mortality of challenged birds receiving the immune modulating actives of the disclosed composition was held to a level not significantly different than that of the unchallenged/untreated control group or the challenged/Coban® treated group.

### Protection of Gut

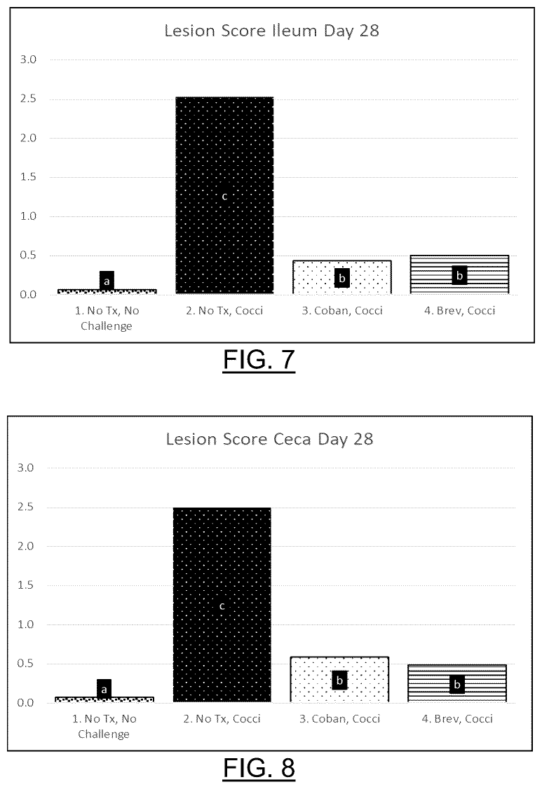

Lesion Scores—Another primary benefit of actives of the disclosed composition is reduced presence and severity of coccidia lesions and damage to the intestinal lining in Eimeria-challenged broiler chickens. Lesion scores in the duodenum, ileum, and ceca are consistently decreased in birds ingesting actives of the disclosed composition, to the same extent as unchallenged control groups and challenged birds treated with leading ionophore products commonly used in poultry production. Lesion score data for the duodenum are provided in FIG. 6. Similar trends were observed in the ileum and ceca as set forth in FIGS. 7 and 8 respectively.

As set forth in FIG. 6 which is a graph illustrating reduction of intestinal lesion score in subject animals fed a composition according to the disclosed inventive concept, intestinal lesion score (Johnson and Reid method) was determined in the duodenum on Day 28 for control birds receiving no Eimeria challenge and no treatment (first bar) and birds challenged with Eimeria on Day 7 but untreated (second bar), challenged and treated with Coban® (third bar), or challenged and treated with the immune modulator of the disclosed composition (fourth bar). As expected, the challenged/untreated group exhibited significantly more lesions due to the Eimeria challenge when compared to the unchallenged control. The challenged/composition-fed group showed no such increase in lesion score. In fact, the lesion score of the challenged/composition-fed group was numerically and statistically lower than that of the unchallenged control group and not significantly different compared to the challenged birds receiving Coban®. Similar lesion score results were found in the ileum as illustrated in FIG. 7 and in the ceca as illustrated in FIG. 8.

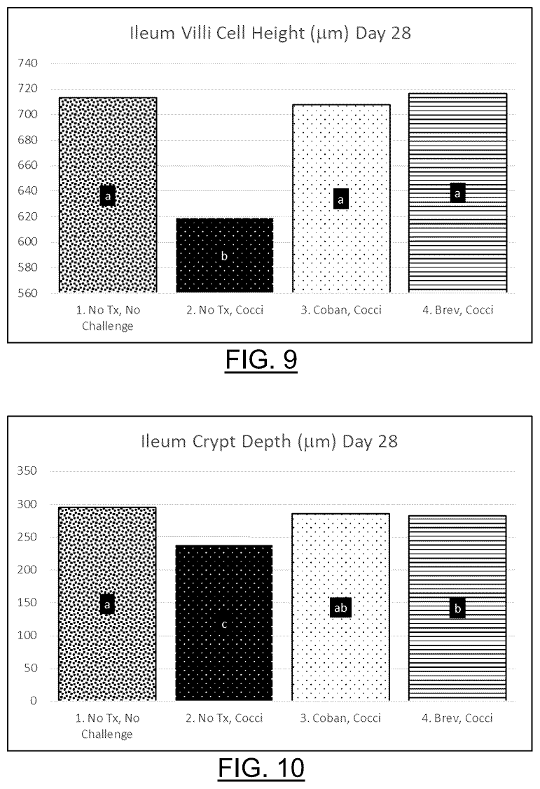

Intestinal Morphology—The immune modulating actives of the disclosed composition also demonstrate positive effects on gut morphology, including restoring or protecting villi height and crypt depth in the ileum following a cocci challenge (FIGS. 9 and 10).

As set forth in FIG. 9 which is a graph illustrating restoration/protection of ileum cell villi height in subject animals fed a composition according to the disclosed inventive concept, cell villi height in the ileum was measured on Day 28 for control birds receiving no Eimeria challenge and no treatment (first bar) and birds challenged with Eimeria on Day 7 but untreated (second bar), challenged and treated with Coban® (third bar), or challenged and treated with the immune modulator of the disclosed composition (fourth bar). The detrimental effects of coccidiosis are clearly seen in the challenged/untreated group which had significantly reduced villi height compared to the unchallenged control group. In contrast, there is no significant difference between the cell villi height measured in birds treated with the disclosed composition as compared to the unchallenged control or in challenged birds treated with Coban®.

As set forth in FIG. 10 which is a graph illustrating restoration/protection of ileum crypt depth in subject animals fed a composition according to the disclosed inventive concept, ileum crypt depth was measured on Day 28 for control birds receiving no Eimeria challenge and no treatment (first bar) and birds challenged with Eimeria on Day 7 but untreated (second bar), challenged and treated with Coban® (third bar), or challenged and treated with the immune modulator of the disclosed composition (fourth bar). Crypt depth in birds fed the actives of the disclosed composition was significantly improved compared to the challenged/untreated group and was not statistically different than for challenged birds treated with Coban®.

As set forth in FIG. 11 which is a graph illustrating restoration/protection of ileum villi height to crypt depth ratios in subject animals fed a composition according to the disclosed inventive concept, which were measured on Day 28 for control birds receiving no Eimeria challenge and no treatment (first bar) and birds challenged with Eimeria on Day 7 but untreated (second bar), challenged and treated with Coban® (third bar), or challenged and treated with the immune modulator of the disclosed composition (fourth bar). Crypt depth ratios in birds fed the actives of the disclosed composition was significantly improved compared to the challenged/untreated group and was only slightly greater than for challenged birds treated with Coban®.

### Secondary Benefits of the Inventive Composition Reduction of Oocysts

Multiple studies demonstrate that the actives of the disclosed composition reduce E. acervulina in the loop of the small intestine area, E. maxima in the jejunum, and E. tenella in the ceca.

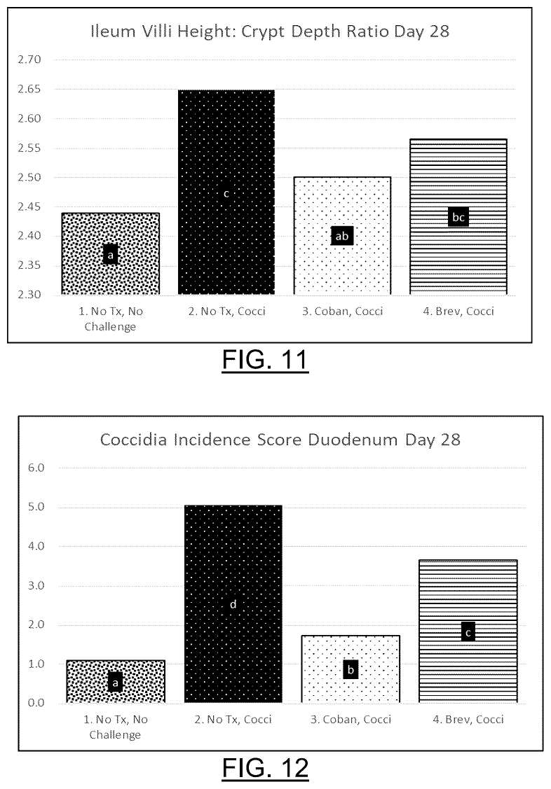

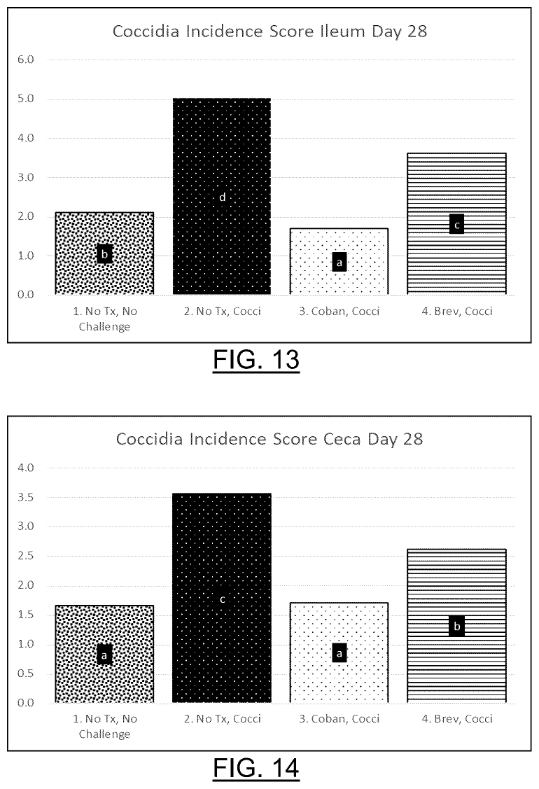

As set forth in FIGS. 12, 13, and 14 which are graphs illustrating reduction of Eimeria in the broiler gut in subject animals fed a composition according to the disclosed inventive concept, coccidia count scores in the duodenum, the ileum, and the ceca respectively are shown confirming trends for all intestinal regions. The coccidia count score was determined on Day 28 for control birds receiving no Eimeria challenge and no treatment (first bar) and birds challenged with Eimeria on Day 7 but untreated (second bar), challenged and treated with Coban® (third bar), or challenged and treated with the immune modulator of the disclosed composition (fourth bar). Actives of the disclosed composition provided a significant reduction in the coccidia score relative to challenge/untreated birds but did not reduce the score to levels comparable with either the unchallenged control or the challenged birds treated with Coban®.

### Second Study

A second study was undertaken to determine the response and efficacy of a dried bacterial biomass feed ingredient incorporated at a specific amount into a commercial-type corn-soybean diet and fed to floor-pen raised broilers. The second study was undertaken over a 28-day period, from Day 1 to Day 28.

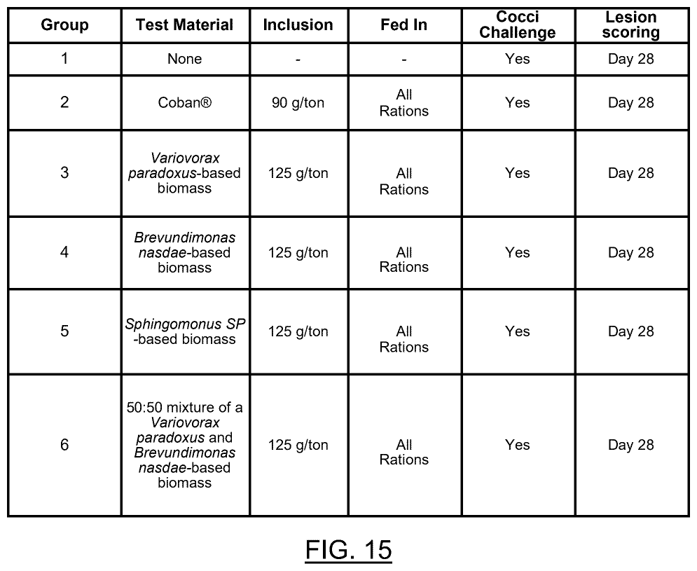

The animals were raised under a disease challenge environment (cocci-challenge+built-up litter). For the study and as set forth in FIG. 15, six groups were established, including a first group that received no feed additive ingredient, a second group treated with the anti-coccidia drug Coban® (Elanco), a third group fed a Variovorax-based biomass, a fourth group fed a Brevundimonas-based biomass, a fifth group fed a Sphingomonas-based biomass, and a sixth group fed a 50:50 mixture of a Variovorax and Brevundimonas-based biomass. The feed included corn and soybean meal rations with normal nutritional formulations. No coccidiostat (except for treatment with Coban® provided to the second group) or ABF (antibiotic free products) were administered during the study. No coccidiosis vaccine was administered at the hatchery or during the course of the study.

### Second Study

A total of 1,800 mixed sex broiler chicks were obtained within twelve hours of hatching from fecal contaminated flocks at a commercial hatchery on Day 0 (hatch and placement day). A number of mixed-sex broiler chicks (50:50 sex ratio) were randomly assigned on Day 0 by individual weights to one of several test group pens, each with replicates. Only antibiotic-free birds were sourced, and no coccidiosis vaccine was administered at the hatchery or at any time during the study. Chicks were evaluated upon receipt for signs of disease or other complications that could affect study outcome. Weak birds were humanely sacrificed. Birds were not replaced during the study.

Following examination, chicks were weighed and allocated to pens for the various treatment groups using a randomized block design. Weight distribution across the treatment groups was assessed prior to feeding by comparing the individual test groups' standard deviations of the mean against that of the control group. Weight distribution across the groups was considered acceptable for this study when differences between control and test groups were within one standard deviation.

All birds received nutritionally adequate diets as pellets (crumbled pellets for Days 1-14). Birds were fed their respective treatment diets ad libitum from day of hatch to 28 days of age. (The typical average market age is 42 days.) Birds were raised on built-up litter to further mimic stress conditions typically experienced in poultry production.

All diets were offered ad libitum without restrictions to full-fed consumption, except for an 8-hour fasting period prior to cocci-challenge on Day 7 when all birds assigned to the challenge test groups received oocyst-inoculated feed containing a mixture of Eimeria acervulina, Eimeria maxima, and Eimeria tenella. Dietary requirements for protein, lysine, methionine, methionine+cystine, arginine, threonine, tryptophan, total phosphorus, available phosphorus, total calcium, dietary sodium, and dietary choline were met by adjusting the concentrations of corn and soybean meal ingredients, as well as other minor ingredients commonly used in poultry production.

Throughout the study, birds were observed at least three times daily for overall health, behavior, and evidence of toxicity. Pens were monitored for environmental conditions, including temperature, lighting, water, feed, litter condition, and unanticipated house conditions/events. Pens were checked daily for mortality. Examinations were performed on all broilers found dead or moribund. Mortalities were recorded (date and weight) and examined (both internal and external body mass).

Cocci-Challenge—On Day 7, all birds assigned to the challenge test groups received oocyst-inoculated feed containing a mixture of Eimeria acervulina, Eimeria maxima, and Eimeria tenella. Adequate feed was precisely weighed and provided to birds to consume at the rate of 100% fill-capacity on average. Prior to the challenge, all birds were starved for eight hours. Inoculated feed was provided to the birds. Following a specific time, all remaining inoculated feed was removed and weighed to assure equal consumption per pen and per bird. The quantity of feed (both placed and withdrawn) was recorded on each pen's feed record.

### Second Study Evaluation

Differences between groups were evaluated at P<0.05, employing Treatment×Replicate RCB (Randomized Complete Block) design. Particularly and as noted, “Significance (P<0.05)” refers to means within a column without a common superscript are significantly different (P<0.05), as determined by Duncan's New Multiple Range Test (MRT 1955).

The relevant evaluation results from the second study are set forth in the following chart and are referenced below by individual graphically represented figures:

| | A | B | C | D | E | F |
|---|---|---|---|---|---|---|
| Means | | | | | | |
| 1. No Tx, Cocci | 1.498 | 15.000 | 1294.183 | 76.196 | 2.075 | 1.683 |
| 2. Coban, Cocci | 1.403 | 3.750 | 1443.182 | 75.493 | 0.317 | 0.325 |
| 3. VP, Cocci | 1.461 | 10.833 | 1339.628 | 75.446 | 1.508 | 1.300 |
| 4. Brev, Cocci | 1.409 | 5.417 | 1433.982 | 76.005 | 1.033 | 0.958 |
| 5. SG, Cocci | 1.456 | 7.917 | 1375.182 | 75.772 | 1.325 | 1.200 |
| 6. VP + Brev, Cocci | 1.435 | 5.833 | 1405.136 | 75.826 | 0.942 | 1.017 |
| Significance | | | | | | |
| (P < 0.05) | | | | | | |
| 1. No Tx, Cocci | d | c | d | a | e | d |
| 2. Coban, Cocci | a | a | a | a | a | a |
| 3. VP, Cocci | c | b | c | a | d | c |
| 4. Brev, Cocci | ab | a | a | a | b | b |
| 5. SG, Cocci | c | ab | bc | a | c | c |
| 6. VP + Brev, Cocci | bc | a | ab | a | b | b |

KEY:
A. Feed Conversion Ratio Corrected Days 1-28
B. % Mortality Days 1-28
C. Avg Pen Body Weight (g) Day 28
D. Feed Intake (g/bird/day) Day 28
E. Lesion Score Duodenum Day 28
F. Lesion Score Ileum Day 28
G. Coccidia Incidence Score Small Intestine Day 28

As noted above, the test subjects were divided into six groups. Members of the first group received no feed additive ingredient in its food regimen. Members of the second group were given feed that included the anti-coccidia drug Coban® (Elanco) according to the manufacturer's instructions. Members of the third group were given feed containing a Variovorax-based biomass. Members of the fourth group were given feed containing a Brevundimonas-based biomass. Members of the fifth group were given feed containing a Sphingomonas-based biomass. Members of the sixth group were given feed containing a 50:50 mixture of a Variovorax and Brevundimonas-based biomass.

All data points were analyzed at the 5% level of probability, including composite weighted average of entire pen, feed: gain and mortality. Birds were evaluated in terms of physical live performance and digestive health. The six groups were evaluated over a 28-day period for body weight, feed conversion ratio (FCR) (corrected for mortality), mortality, body weight gain, and average weight gain.

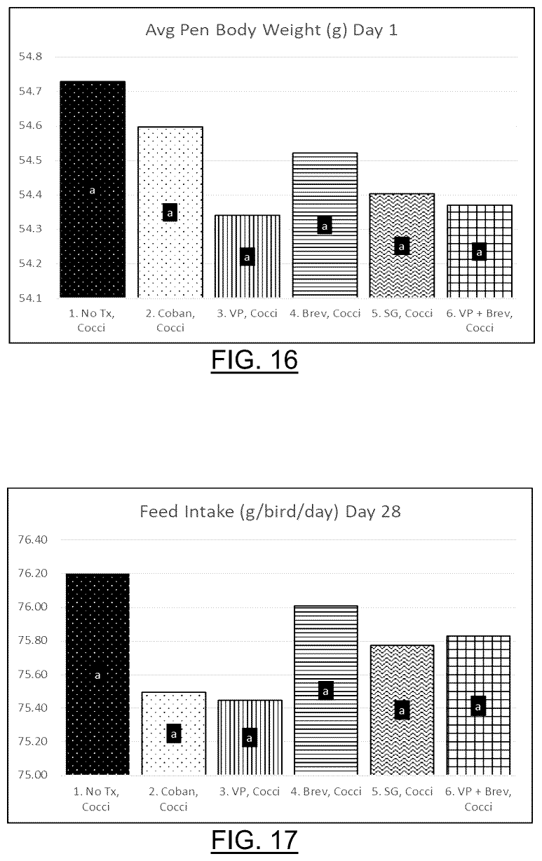

Day 1 Body Weight Evaluation—Referring to FIG. 16, Average Body Weight at Day 1 is illustrated. Body weights are not statistically different among groups at this stage, ranging from a high weight of 54.727 g for Group 1 to a low weight of 54.339 g for Group 3:

| | Avg Pen Body |
|---|---|
| | Weight (g) |
| Means | Day 1 |
| 1. No Tx, Cocci | 54.727 |
| 2. Coban, Cocci | 54.595 |
| 3. VP, Cocci | 54.339 |
| 4. Brev, Cocci | 54.521 |
| 5. SG, Cocci | 54.402 |
| 6. VP + Brev, Cocci | 54.369 |
| Significance (P < 0.05) | |
| 1. No Tx, Cocci | a |
| 2. Coban, Cocci | a |
| 3. VP, Cocci | a |
| 4. Brev, Cocci | a |
| 5. SG, Cocci | a |
| 6. VP + Brev, Cocci | a |

Feed Intake—The graph set forth in FIG. 17 illustrates a feed intake comparison in subject animals fed a composition according to the disclosed inventive concept. Feed intake was measured at Day 28 for Group 1 (first bar), for Group 2 (second bar), for Group 3 (third bar), for Group 4 (fourth bar), for Group 5 (fifth bar), and for Group 6 (sixth bar). Feed intake was highest in challenged/untreated birds. However, there were no statistical differences in feed intake for any of the groups.

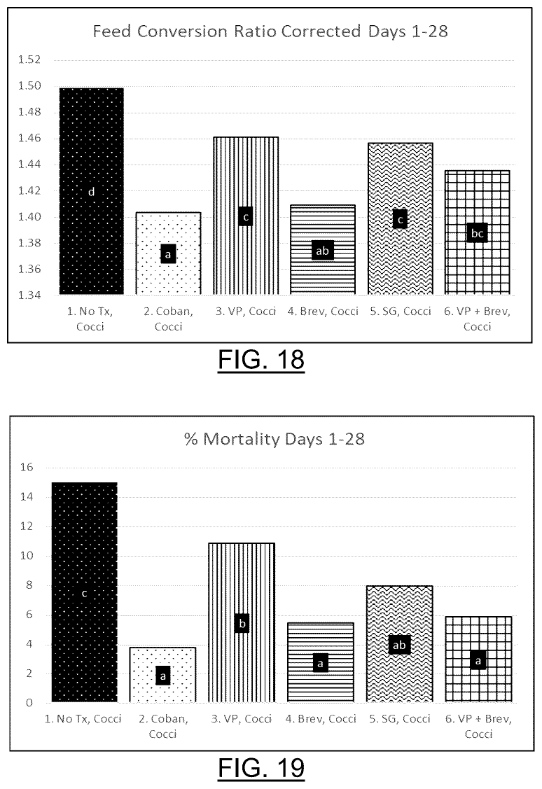

Feed Conversion Rates Corrected—FCR was recorded on Days 1-28 as shown in FIG. 18. Group 2, which received feed that included Coban®, had the lowest FCR, while Group 4, which received feed containing a Brevundimonas-based biomass had the second lowest rate. Starting on Day 8 and continuing throughout the study duration, improvements in FCR for Group 4 were not statistically different from Group 2, demonstrating that the Brevundimonas-based biomass in feed performed as well as a long-standing, commercially available coccidiosis treatment, such as Coban®. Groups 2 and 4 had statistically lower FCR compared to Group 1, which received no feed additive ingredient in its food regimen.

Mortality Percentage—Mortality rates were recorded on Days 1-28 as illustrated in FIG. 19. Mortality rates were lowest for members of the second group, which received feed that included Coban® and for members of the fourth group, which received feed containing a Brevundimonas-based biomass as well as for Group 6, which received feed containing a 50:50 mixture of a Variovorax and Brevundimonas-based biomass.

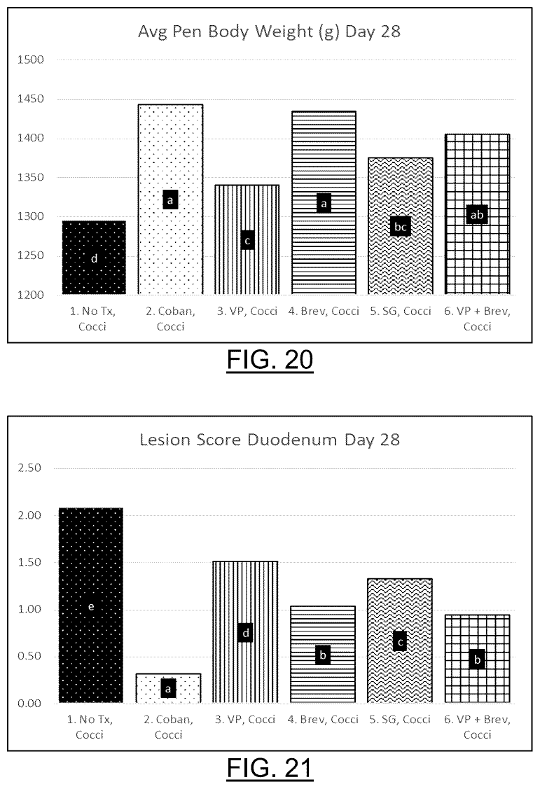

Average Pen Body Weight—Average pen body weight was recorded on Day 28 and is set forth in FIG. 20. Weight gain was most pronounced for the second group, which received feed that included Coban® but was followed closely by weight gain in members of the fourth group, which received feed containing a Brevundimonas-based biomass. Members of Group 6, which received feed containing a 50:50 mixture of a Variovorax and Brevundimonas-based biomass also showed noteworthy weight gain.

Lesion Scores—Gross necropsy and lesion scoring were performed. Birds were selected, sacrificed, weighed, and examined for the presence and degree of duodenal and coccidia lesions. Damage scores were assessed and recorded.

With respect to the duodenum lesion scores for Day 28, the lowest score was evident with the second group, which received Coban®, followed by groups 4 given feed containing a Brevundimonas-based biomass and 6 given feed containing a 50:50 mixture of a Variovorax and Brevundimonas-based biomass. These results are illustrated in FIG. 21.

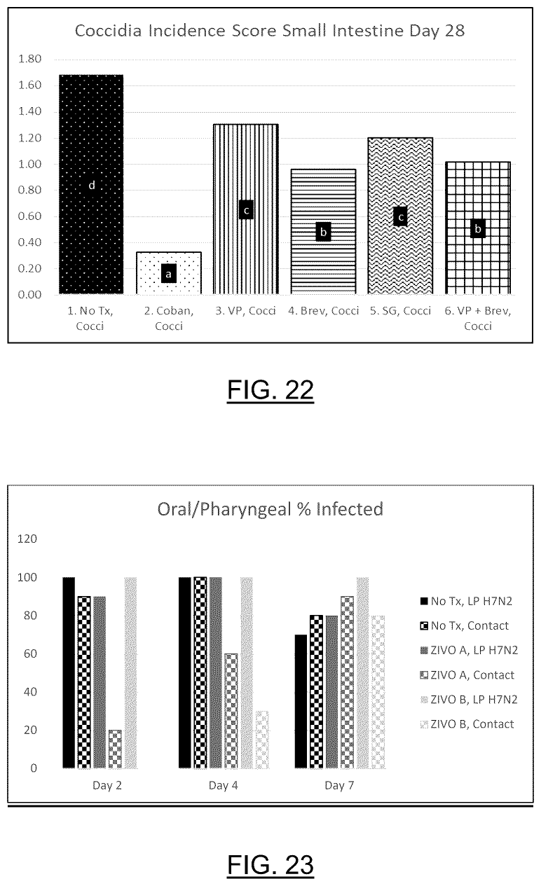

With respect to the coccidia lesion incidences score of the small intestine for Day 28, the lowest score was again experienced by the second group, a statistically significant reduction compared to all other groups, followed by the fourth and sixth groups. These results are illustrated in FIG. 22.

### Third Study

Applicants' first Low Pathogenicity Avian Influenza (LPAI) study generated evidence supporting the applications of the disclosed composition in mitigating the spread of the virus among poultry. According to the third study, Applicant tested algae containing multiple species of bacteria (referred to as Product A [Prod A]) and Product B [Prod B], a Brevundimonas bacterial strain. The focus of the third (and fourth) studies is on the H7N2 virus, an influenza virus typically seen circulating in birds. The avian influenza virus is frequently referred to as “bird flu” or “avian flu.” It does not normally infect humans although rare cases of human infection have occurred in the event of direct contact with infected birds.

The brief results of the third study are set forth below and are set forth in FIG. 23.

% infected

| Oral/Pharyngeal | Day 2 | Day 4 | Day 7 |
|---|---|---|---|
| No Tx, LP H7N2 | 100 | 100 | 70 |
| No Tx, Contact | 90 | 100 | 80 |
| ZIVO A, LP H7N2 | 90 | 100 | 80 |
| ZIVO A, Contact | 20 | 60 | 90 |
| ZIVO B, LP H7N2 | 100 | 100 | 100 |
| ZIVO B, Contact | 0 | 30 | 80 |

### Third Study—Treatment Method

Poultry & Eggs. Day of hatch, specific pathogen free (SPF) white leghorn chickens were obtained from AVSBio (Norwich, CT). All birds were housed under negative pressure in glove port isolators (Allentown, LLC., Allentown, NJ) throughout the experiment and offered feed and water ad libitum. At the end of the study, all birds were euthanized using American Veterinary Medical Association (AVMA)-approved procedures (AVMA Guidelines on Euthanasia, 2020).

Virus. Titers of viral stocks of the low-path isolate A/Chicken/Maryland/MinMah/2004 H7N2) were determined. Ten 10-fold dilutions were created in BHI (Becton, Dickinson and Company, Franklin Lakes, NJ) containing 10,000 IU/mL of penicillin and 10,000 μg/mL of streptomycin (Lonza Group, Walkersville, MD) and allowed to incubate at 23 C (+/−2 C) for 60 minutes (+/−5 min). Thirty, ten-day-old embryonating SPF chicken eggs (three per dilution) were inoculated via the chorioallantoic sac (CAS) route and incubated at 37 C (+/−1 C) for 7 days. Eggs were chilled at 4 C (+/−2 C) for 24 hr (+/−5 hr) prior to evaluation. Chick amniotic fluids (CAF) were collected from each egg and tested for the ability to hemagglutinate chicken red blood cells (Killian, 2008). Titers were calculated as mean embryo infectious doses (EID50) (Reed and Muench, 1938) using the reciprocal of the highest dilution of virus at which 50% of the eggs were infected.

Evaluation of interventions. Day of hatch, SPF chicks were divided into groups of according to Table 1 and Table 2. Based upon assigned treatments, birds were offered a formulated base ration that either contained an intervention or did not. Birds were held for 3 weeks in isolation during this intervention loading period. On Day 25, feed was weighed, and all birds were weighed. This data was used to determine weight gain and feed conversion during the infection period.

TABLE 1
Experimental plan: Direct Challenge

| | | | | Challenge at 3 Weeks of Age |
|---|---|---|---|---|
| | | | | with |
| | | Number of | | Chicken/Maryland/MinMah/04 |
| | | One Day | | (CK/MD/MinMah/04 H7N2) |
| | | Old SPF | Duration of | 10⁶EID₅₀/bird/0.1 ml by the |
| Group | Treatment | Chickens | Treatment | intrachoanal route |
| 1 | None | 20 | N/A | No |
| 2 | None | 20 | N/A | Yes |
| 3 | Product A | 20 | 3 weeks | Yes |
| 4 | Product B | 20 | 3 weeks | Yes |

Direct challenge (Table 1). All chickens in groups 2, 3 and 4 were infected with 10⁶EID₅₀/bird/0.1 ml H7N2 LPAIV strain by the intrachoanal route. Response to infection as determined by the observation of clinical signs and analysis of real-time RT-PCR relative oral/pharyngeal and cloacal titers on days 2, 4, & 7 post inoculation (PI). For clinical signs, the birds were scored as follows: 0=no clinical signs; 1=mild to moderate respiratory signs in chickens; 2=moderate to severe respiratory signs including depression, decreased feed and water intake and neurological signs; 3=and death. On day 7 PI, feed and individual birds were weighed. The weight of any additional feed was captured throughout the trial. Oral/pharyngeal (O/P) and cloacal swabbings were tested using the AIV Matrix real-time RT-PCR assay (Spackman et al., 2002¹) utilizing a standard curve of each virus to determine relative viral titers for each swab.

TABLE 2
Experimental plan: Horizontal Spread

| | | | | Challenge at 3 Weeks of Age |
|---|---|---|---|---|
| | | Number | | with |
| | | of One | | Chicken/Maryland/MinMah/04 |
| | | Day Old | | (CK/MD/MinMah/04 H7N2) |
| | | SPF | Duration of | 10⁶EID₅₀/bird/0.1 ml |
| Group | Treatment | Chickens | Treatment | by the intrachoanal route |
| 5 | Product A | 10 | 4 weeks | Not Challenged |
| 6 | Product A | 10 | 4 weeks | 10 + 10 sentinels from group 5 |
| | | | | (sentinels added @ 12 hr post- |
| | | | | challenge) |
| 7 | Product B | 10 | 4 weeks | Not Challenged |
| 8 | Product B | 10 | 4 weeks | 10 + 10 sentinels from group 7 |
| | | | | (sentinels added @ 12 hr post- |
| | | | | challenge) |
| 9 | None | 10 | N/A | Not Challenged |
| 10 | None | 10 | N/A | 10 + 10 sentinels from group 9 |
| | | | | (sentinels added @ 12 hr post- |
| | | | | challenge) |

Horizontal Spread (Table 2). All chickens in groups 6, 8 and 10 were infected with 10⁶EID₅₀/bird/0.1 ml of an H7N2 LPAIV strain by the intrachoanal route. At 12 hrs post infection, birds from groups 5, 7 and 9 were added to the appropriate cages as sentinel birds to assess the ability of the interventions to reduce the impact of horizontal spread. Response to infection was determined by the observation of clinical signs and analysis of real-time RT-PCR relative oral/pharyngeal and cloacal titers on days 2, 4, & 7 PI. For clinical signs, the birds were scored as follows: 0=no clinical signs; 1=mild to moderate respiratory signs in chickens; 2=moderate to severe respiratory signs including depression, decreased feed and water intake and neurological signs; 3=and death. Oral/pharyngeal (O/P) and cloacal swabbings were tested using the AIV Matrix real-time RT-PCR assay (Spackman et al., 2002) utilizing a standard curve of each virus to determine relative viral titers for each swab.

RNA Extraction and Real-Time RT-PCR. Viral RNA was extracted from O/P and cloacal swabs using the Mag-MAX™ Pathogen Isolation Kit (Ambion, Inc., Austin, TX) with the automated King Fisher 96 (Thermo Fisher Scientific, Waltham, MA) in a 96-well format. To determine the estimated viral titer in each sample, each plate contained the respective test virus dilutions (10⁻¹ to 10⁻⁶) in duplicate to establish a standard curve. Quantitative real-time RT-PCR was performed using the Applied Biosystems 7500 Fast Real-Time PCR System (Foster City, CA) and Ambion AgPath-ID™ (Ambion, Inc., Austin, TX) chemistry along with primers and probes targeting a highly conserved region of the matrix gene (Spackman et al., 2002).

Statistical Analysis. Statistical significance between pre-challenge HI antibody titres were determined by ANOVA using the Tukey's multiple comparison tests. Statistical significance between post-challenge percent morbidity and mortality figures were determined by chi-square test. Statistical analyses of HI titers and mortality figures were conducted with using the PH Stat (version 4) program (Pearson Education Inc., London, UK) for Excel. All statistical tests were performed using P<0.05. Superscript lowercase letters in tables and graphs indicate statistical significance between aged-matched groups. Statistical groups are denoted in tables and graphs by lowercase letters. Groups apply to values in the same table column.

Differences in shedding levels between treatment groups by the same swab type and day post-inoculation were tested for significance by one-way ANOVA, Tukey's test with JMP Pro (version 17.2.0). Statistical groups are denoted in graphs by lowercase letters. A P-value of ≤0.05 was considered significant.

### Third Study Evaluation

In brief, the horizontal spread of the virus on Day 2 was completely prevented in the Product B/Contact group and statistically lower in the Product A/Contact group (20% infected). In contrast, 100% of the sentinel birds in the None/Contact group were infected on Day 2. On Day 4, the sentinel birds in the Product A/Contact and Product B/Contact groups continued to have lower rates of infection compared to the None/Contact group (60%, 30%, and 100%, respectively). This positive effect waned by Day 7 post-exposure.

More particularly, prior to viral challenge, all birds were successfully raised to 25 days of age without issues related to feed rejection. Initial bird weights across groups were consistent, and while differences in weight gain percentages were observed, no clear trends emerged. Feed conversion ratios and feed consumption remained unaffected by the additives. Following direct viral challenge (Groups 1-4) Product A treatment reduced O/P viral shedding at Days 4 and 7 post-challenge compared to infected controls, though differences were often numerical rather than statistical. Cloacal swabs showed minimal viral detection, limiting trend analysis. In the evaluation of horizonal transmission (Groups 5-10), viral spread was significantly reduced in the product B and Product A contact groups compared to controls on Days 2 and 4, though this effect diminished by Day 7. Cloacal swab data showed infrequent viral detection, and no remarkable statistical differences were observed. Overall, Product A and Product B treatments demonstrated the potential to mitigate viral shedding and horizontal transmission, though further studies to optimize treatment formulation and dosing are needed.

### Fourth Study

Applicants' fourth LPAI study generated evidence also supporting the effectiveness of the disclosed composition in in mitigating the spread of the virus among poultry. According to the fourth study, Applicant tested a composition which included up to four bacterial species: Brevundimonas sp., Microbacterium sp., Sphingomonas sp., and Variovorax sp. Three specific compositions were tested as follows:

| | | |
|---|---|---|
| ZIVO-1 | bac biomass V | 600 g/ton |
| ZIVO-2 | bac biomass V + B | 1200 g/ton (600 g |
| | | each) |
| ZIVO-3 | bac | 2400 g/ton (600 g |
| | biomass V + B + M + S | each) |

$$Wherein V = Variovorax , V + B = Variovorax + Brev , and$$

$$V + B + M + S = Variovorax + Brev + Microbacterium + Sphingomonas$$

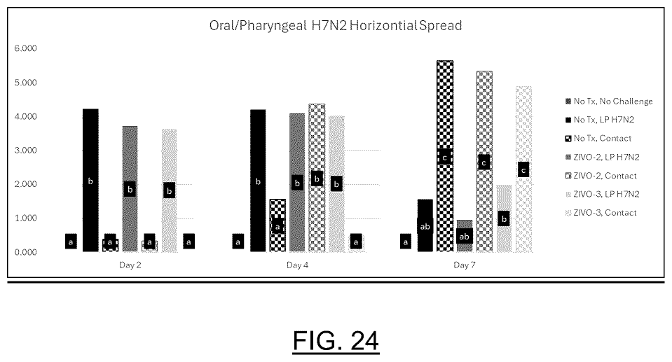

The brief results of the fourth study are set forth below and are set forth in FIG. 24.

Copies of H7N2

| Oral/Pharyngeal | Day 2 | Day 4 | Day 7 |
|---|---|---|---|
| No Tx, No Challenge | 0.000 | 0.000 | 0.000 |
| No Tx, LP H7N2 | 4.219 | 4.204 | 1.557 |
| No Tx, Contact | 0.381 | 1.560 | 5.639 |
| ZIVO-2, LP H7N2 | 3.718 | 4.097 | 0.954 |
| ZIVO-2, Contact | 0.324 | 4.356 | 5.321 |
| ZIVO-3, LP H7N2 | 3.649 | 4.030 | 1.989 |
| ZIVO-3, Contact | 0.000 | 0.492 | 4.871 |
| No Tx, No Challenge | a | a | a |
| No Tx, LP H7N2 | b | b | ab |
| No Tx, Contact | a | a | c |
| ZIVO-2, LP H7N2 | b | b | ab |
| ZIVO-2, Contact | a | b | c |
| ZIVO-3, LP H7N2 | b | b | b |
| ZIVO-3, Contact | a | a | c |

### Fourth Study—Treatment Method

Poultry & Eggs. Day of hatch, specific pathogen free (SPF) white leghorn chickens were obtained from AVSBio. All birds were housed under negative pressure in glove port isolators for throughout the experiment and offered feed and water ad libitum.

Virus. Current titers of viral stocks of the low-path isolate A/Chicken/Maryland/MinMah/2004 (H7N2) were determined as described (Woolcock, 2008) with some modifications. Briefly, ten, 10-fold dilutions of a virus were created in BHI (Becton, Dickinson and Company) containing 10,000 IU/mL of penicillin and 10,000 μg/mL of streptomycin (Lonza Group) and allowed to incubate at 23 C (+/−2 C) for 90 minutes (+/−5 min). Thirty, ten-day-old embryonating SPF chicken eggs (three per dilution) were inoculated via the CAS route and incubated at 37 C (+/−1 C) for 7 days. Eggs were chilled at 4 C (+/−2 C) for 24 hr (+/−5 hr) prior to evaluation. CAF were collected from each egg and tested for the ability to hemagglutinate chicken red blood cells (Killian, 2008). Titers were calculated as mean embryo infectious doses (EID50) (Reed and Muench, 1938) using the reciprocal of the highest dilution of virus at which 50% of the eggs are infected.

Evaluation of Applicants' interventions. Day of hatch, SPF chicks were divided into groups of according to Table 1 and Table 2. Based upon assigned treatments, birds were offered a formulated base ration that contains an intervention or does not. Birds were held for 3 weeks in isolation during this intervention loading period. Between 3 and 4 weeks of age (3 weeks/4 days max), feed were weighed, and all birds were weighed. This data were used to determine weight gain and feed conversion during the infection period.

Direct challenge (Table 1): All chickens in groups 1b, 2, 3 and 4 were infected with 10⁶EID50/bird/0.1 ml H7N2 LPAIV strain by the intrachoanal route. Response to infection were determined by the observation of clinical signs and analysis of real-time RT-PCR relative O/P titers on days 2, 4, & 7 PI. For clinical signs, the birds were scored as follows: 0=no clinical signs; 1=mild to moderate respiratory signs in chickens; 2=moderate to severe respiratory signs including depression, decreased feed and water intake and neurological signs; 3=and death. On day 7 PI, feed and individual birds were weighed. The weight of any additional feed were captured throughout the trial. Birds unable to access food and water due to severity of disease were euthanized using American Veterinary Medical Association (AVMA)-approved procedures (AVMA Guidelines on Euthansia, 2020). Oropharyngeal (O/P) swabbings were tested using the AIV Matrix real-time RT-PCR assay (Spackman et al., 2002) utilizing a standard curve of the virus to determine relative viral titers for each swab. Birds were observed until day 7 PI before being euthanized.

Horizontal Spread (Table 2). All chickens in groups 5, 7 and 9 were infected with 10⁶EID50/bird/0.1 ml of an H7N2 LPAIV strain by the intrachoanal route. At 12 hrs post infection, birds from groups 5, 7 and 9 be added to the appropriate cages (6, 8 and 10, respectively) to assess the ability of the interventions to reduce the impact of horizontal spread. Response to infection were determined by the observation of clinical signs and analysis of real-time RT-PCR relative oropharyngeal titers on days 2, 4, & 7 PI. For clinical signs, the birds were scored as follows: 0=no clinical signs; 1=mild to moderate respiratory signs in chickens; 2=moderate to severe respiratory signs including depression, decreased feed and water intake and neurological signs; 3=and death. Birds unable to access food and water due to severity of disease were euthanized using American Veterinary Medical Association (AVMA)-approved procedures (AVMA Guidelines on Euthansia, 2020). Oropharyngeal (O/P) swabbings were tested using the AIV Matrix real-time RT-PCR assay (Spackman et al., 2002) utilizing a standard curve of the virus to determine relative viral titers for each swab.

RNA Extraction and Real-Time RT-PCR: Viral RNA were extracted from O/P swabs using the Mag-MAX™ Pathogen Isolation Kit (Ambion, Inc., Austin, TX) with the automated King Fisher 96 (Thermo Fisher Scientific, Waltham, MA) in a 96-well format. To determine the estimated viral titer in each sample, each plate contained the respective test virus dilutions (10⁻¹ to 10⁻⁶) in duplicate to establish a standard curve. Quantitative real-time RT-PCR were performed using the Applied Biosystems 7500 Fast Real-Time PCR System (Foster City, CA) and Ambion AgPath-ID™ (Ambion, Inc., Austin, TX) chemistry along with USDA Matrix primers and probe (Spackman et al., 2002).

TABLE 1
Experimental plan: Direct Challenge

| | | | | Challenge at 3 | |
|---|---|---|---|---|---|
| | | | | Weeks of Age with | |
| | | Number | | Chicken/Maryland/ | |
| | | of One | | MinMah/04 | Duration of |
| | | Day Old | Duration | (CK/MD/MinMah/04 | Observation |
| | | SPF | of | H7N2) | Period Post |
| Group | Treatment | Chickens | Treatment | 10⁶EID₅₀/bird/0.1 ml^{a} | Challenge |
| 1A | Control | 10 | N/A | No | 1 week |
| | Diet: None | | | | |
| 1B | Control | 10 | N/A | Yes | 1 week |
| | Diet: None | | | | |
| 2 | Zivo-1: | 20 | 4 weeks | Yes | 1 week |
| | Bacterial | | | | |
| | Biomass V | | | | |
| 3 | Zivo-2: | 20 | 4 weeks | Yes | 1 week |
| | Bacterial | | | | |
| | Biomass V + B | | | | |
| 4 | Zivo-3: | 20 | 4 weeks | Yes | 1 week |
| | Bacterial | | | | |
| | Biomass V + | | | | |
| | B + M + S | | | | |

^{a}Intrachoanal route of inoculation.

TABLE 2
Experimental plan: Horizontal Challenge

| | | | | Challenge at 3 | |
|---|---|---|---|---|---|
| | | | | Weeks of Age with | |
| | | Number | | Chicken/Maryland/ | |
| | | of One | | MinMah/04 | Duration of |
| | | Day Old | Duration | (CK/MD/MinMah/04 | Observation |
| | | SPF | of | H7N2) | Period Post |
| Group | Treatment | Chickens | Treatment | 10⁶EID₅₀/bird/0.1 ml^{a} | Challenge |
| 5 | Control | 10 | N/A | Yes, directly | 1 week |
| | Diet: None | | | challenged, moved | |
| | | | | to cage 6 @12 hrs | |
| 6 | Control Diet: | 10 | N/A | No, exposed to Group | 1 week |
| | | | | 5 | |
| | None | 10 | N/A | No | 1 week |
| 7 | Zivo-2: | 10 | 4 weeks | Yes, directly | 1 week |
| | Bacterial | | | challenged, moved | |
| | Biomass V + B | | | to cage 8 @12 hrs | |
| 8 | Zivo-2: | 10 | 4 weeks | No, exposed to | 1 week |
| | Bacterial | | | Group 7 | |
| | Biomass V + B | | | | |
| 9 | Zivo-3: | 10 | 4 weeks | Yes, directly | 1 week |
| | Bacterial | | | challenged, moved | |
| | Biomass V + | | | to cage 10 @12 hrs | |
| | B + M + S | | | | |
| 10 | Zivo-3: | 10 | 4 weeks | No, exposed to | 1 week |
| | Bacterial | | | Group 9 | |
| | Biomass V + | | | | |
| | B + M + S | | | | |

^{a}Intrachoanal route of inoculation.

TABLE 3
Experimental plan: Sample Collection Post Challenge

| | | Challenge at 3 | | | Collection |
|---|---|---|---|---|---|
| | | Weeks of Age with | | | of Bird |
| | | Chicken/ | Scoring of | Collection of | Body |
| | | Maryland/MinMah/ | Clinical | Oropharyngeal | Weights |
| | | 04 (CK/MD/MinMah/ | Disease | Swabs (days | and Feed |
| | | 04 H7N2)10⁶EID50/ | (days post | post | (days post |
| Grp | Treatment | bird/0.1 ml^{a} | challenge)^{a} | challenge) | challenge) |
| 1A | Control Diet: | No | 2, 4, 7 | 2, 4, 7 | 0, 7 |
| | None | | | | |
| 1B | Control Diet: | Yes | 2, 4, 7 | 2, 4, 7 | 0, 7 |
| | None | | | | |
| 2 | Zivo-1: Bacterial | Yes | 2, 4, 7 | 2, 4, 7 | 0, 7 |
| | Biomass V | | | | |
| 3 | Zivo-2: Bacterial | Yes | 2, 4, 7 | 2, 4, 7 | 0, 7 |
| | Biomass V + B | | | | |
| 4 | Zivo-3: Bacterial | Yes | 2, 4, 7 | 2, 4, 7 | 0, 7 |
| | Biomass V + B + | | | | |
| | M + S | | | | |
| 5 | Control Diet: | Yes, directly | 2, 4, 7 | 2, 4, 7 | 0, 7 |
| | None | challenged, moved to | | | |
| | | cage 6 @12 hrs | | | |
| 6 | Control Diet: | No, exposed to Group | 2, 4, 7 | 2, 4, 7 | 0, 7 |
| | None | 5 | | | |
| 7 | Zivo-2: Bacterial | Yes, directly | 2, 4, 7 | 2, 4, 7 | 0, 7 |
| | Biomass V + B | challenged, moved to | | | |
| | | cage 8 @12 hrs | | | |
| 8 | Zivo-2: Bacterial | No, exposed to | 2, 4, 7 | 2, 4, 7 | 0, 7 |
| | Biomass V + B | Group 7 | | | |
| 9 | Zivo-3: Bacterial | Yes, directly | 2, 4, 7 | 2, 4, 7 | 0, 7 |
| | Biomass V + B + | challenged, moved to | | | |
| | M + S | cage 10 @12 hrs | | | |
| 10 | Zivo-3: Bacterial | No, exposed to | 2, 4, 7 | 2, 4, 7 | 0, 7 |
| | Biomass V + B + | Group 9 | | | |
| | M + S | | | | |

^{a}Birds were observed and evaluated daily with a thorough evaluation of birds occurring on days 2, 4, 7 and 14 post challenge.

### Fourth Study Evaluation

The fourth study demonstrate that the present composition reduced the LPAI virus among poultry. Particularly, the fourth study demonstrated a numerical reduction in viral titers (viral shedding) two days post-infection in infected birds receiving all three formulations of the disclosed composition compared with untreated infected controls. The fourth study also demonstrated a delay in transmission of LPAI on day 2 post-infection when healthy birds fed ZIVO-2 or ZIVO-3 were exposed to infected birds, suggesting a slower and less aggressive spread of disease. ZIVO-3 showed superior efficacy in subsequent time periods. The fourth study successfully demonstrated the efficacy of the present composition and confirmed that the treatment composition is efficacious for mitigating the effects of coccidiosis in broiler chickens, against LPAI.

Overall, in the fourth study it was confirmed that infected birds receiving a mixture of the disclosed composition showed an early significant decrease in viral titers compared with untreated, infected controls, thereby reducing amount of detectable virus that was shed. At the end of the study, although not significant in nature, a numerical decrease in virus was noted in birds receiving the disclosed composition. According to the fourth study, healthy chickens were housed with infected birds, replicating a real-world, high-risk environment for disease transmission. Compared to an untreated control group, birds receiving the present composition that were housed with infected birds experienced a statistically significant delay in viral detection. This observed delay suggests that the present composition limits viral replication within a host. Such favorable results indicate that the composition's active ingredients represent potential preventative measures for reducing the spread of LPAIV in commercial poultry operations and enhancing overall flock health.

### Results—in General

The disclosed inventive feed additive containing a Brevundimonas-based, Microbacterium-based, Sphingomonas-based, and Variovorax-based biomass consistently promoted improved growth performance in broilers receiving a coccidiosis challenge that was not statistically different from that observed for birds given feed that included the anti-coccidia drug Coban®. This outcome holds true over all assessed growth performance characteristics including body weight, feed conversion ratio (FCR), mortality, body weight gain and average weight gain. The positive results obtained with the inventive composition were so observed without risk of drug resistance developing as is often the risk with the use of anti-coccidia drugs.

### I. Results—Agents and Method for Improving Gut Health

The disclosed inventive composition demonstrates improved live performance and digestive health parameters for weight gain, feed efficiency, mortality, intestinal villi cell height, crypt depth, villi height to crypt depth ratio, and intestinal coccidiosis across all age ranges. The disclosed inventive composition is effective in ameliorating the physical effects of environmental stress on live performance that are typically experienced in poultry production.

The disclosed inventive composition demonstrates a positive impact on the gut health of boilers through improved gut morphology under disease stress by improving nutrient uptake and eventual bird growth and overall improved gut integrity. The treatment method and compound may have benefits that go beyond the poultry industry to other animals and possibly to humans.

### II. Results—Use of Brevundimonas Microbes as an Alternative Treatment for Coccidiosis

In an additional study which considered the use of Brevundimonas microbes as an alternative treatment for coccidiosis, analysis of tissue from broiler chickens provided a feed mixture that included the disclosed compound demonstrated a significant maturing of innate immune pathways when compared with sacrificed birds fed a conventional feed mixture without the disclosed compound. Also, in general, kinomic analysis of tissue from the birds provided a feed mixture that included the disclosed compound demonstrated significant enhancement of adaptive immune pathways.

Furthermore, analysis of the broiler tissues from chickens fed the treatment compound according to the regimen set forth above, as well as those not fed the treatment compound, verified that the treated chickens demonstrated an increase in adaptive immune response indicating a more immunologically competent or developed immune system. Significantly, the analysis indicated that treatment with the disclosed compound resulted in a more adaptive-biased immune system in the supplemented birds and further demonstrated an alteration of immunometabolism in gut, liver, and muscle tissue by priming the animal for a more rapid and robust immune response compared to birds receiving a standard, unfortified diet. Comparative analysis of tissues of chickens fed the treatment compound and those not fed the treatment compound verified that the treated chickens demonstrated an altered immune response consistent with the effects of TLR4 modulation.

Advantageously, studies showed that the identified immunomodulatory adaptation does not come at the cost of impaired growth and development, rather metabolic efficiency was facilitated in the latter part of life. By following the method and treatment of the disclosed inventive concept, a more rapid, adaptive immune response that is comparable to a response usually seen in an older bird is possible in young birds.

### III. Results—Natural Feed Composition Containing Brevundimonas for the Promotion of Animal Growth

In general, analysis of the results supports the conclusion that inclusion of the innovative compound as part of a conventional diet leads to a significant increase in growth and weight gain when compared to the control flock.

Moreover, following treatment with the disclosed compound on selected birds, samples of both treated and non-treated birds were examined by gross necropsy which included internal examination. Kinomic analysis of tissues collected from sacrificed birds fed the dietary mixture of the inventive composition and conventional feed confirmed that the biomass alters multiple growth-related pathways as proposed, thus initiating pathway activation.

Further analysis demonstrates that the birds consuming the supplemented diet converted feed into body mass more efficiently than the birds fed the control diet. Birds receiving the diet supplemented with the inventive composition had statistically significant decrease in FCR compared to the untreated control group in the given studies. Perhaps most significantly, while there were no differences in overall carcass yield, the total breast weight, as well as the Pectoralis major (breast fillets) and Pectoralis minor (tender) weights, were increased in the group fed the diet supplemented with the inventive composition disclosed herein.

### IV. Results—Enduring Effects on Coccidiosis Prevention and Treatment Via Animal Feed

The use of the innovative compound in the treatment of coccidiosis-challenged poultry demonstrates an enduring effect on the coccidiosis prevention and treatment by delivery through animal feed whereby the positive effects of treatment persist well beyond the time of product withdrawal unlike the loss of efficacy noted immediately after withdrawal of conventional anticoccidial products. Overall, analysis of birds fed the test material for the first 14 days of life demonstrated changes in the immune system consistent with a bolstered innate immune response thereby providing enduring effects over the life of the animal.

Notably, a significant decrease in the presence and degree of coccidia lesions and damage to the intestinal lining typically experienced following coccidiosis infection was noted.

Upon examination of the sacrificed birds, it was found that the average lesion scores throughout the intestine of poultry treated with the disclosed composition were consistently lower than the scores of sacrificed untreated disease-challenged birds. ileum villi cell height, crypt depth, and the villus height to crypt depth ratio generally showed improvement. Particularly, ileum villi cell height and crypt depths in birds fed the inventive formulations disclosed herein were competitive with coccidiosis-challenged birds treated with conventional antibiotics.

The average oocyst count of the duodenum, mid-gut, and cecum of sample poultry given feed or water having the disclosed inventive composition were lower than the scores of sacrificed untreated disease-challenged birds. The groups treated with the inventive composition had improved mortality compared to challenged, untreated groups.

### V. Results—Enduring Effects on Low Pathogenicity Avian Influenza (LPAI) Prevention and Treatment Via Animal Feed

The use of the innovative compound demonstrated effectiveness against Low Pathogenicity Avian Influenza (LPAI) by delivery through animal feed. Studies demonstrate that the present composition reduced viral titers in infected birds receiving the disclosed composition compared with untreated infected controls. Treatment by use of the present composition also demonstrated a delay in transmission of LPAI when healthy birds were exposed to infected birds indicating a slower and less aggressive spread of disease.

The improvement of the overall health of disease-challenged poultry as a result of being given feed or water containing the disclosed inventive composition was achieved without the use of antibiotics. Overall the inventive composition demonstrates a cost-effective and practical approach to the treatment of disease states in animals.

We claim:

1. A method for increasing protection from disease affecting poultry during the time interval between initial vaccination of the animal and its achieving immunity, the method including the steps of administering to the bird an effective amount of a vaccine together with feeding the bird an effective amount of a composition, the composition being derived from the group consisting essentially of an algal biomass containing Gram-negative bacteria, a supernatant from an algal culture containing an algal organism and Gram-negative bacteria, a bacterial biomass containing Gram-negative bacteria, a lipopolysaccharide-Lipid A compound derived from a bacterial strain, a cellular component, fraction or extract derived from a bacterial strain containing lipopolysaccharide-Lipid A, and a lipopolysaccharide-Lipid A derivative from a bacterial strain, the Gram-negative bacteria being selected from the group consisting of Brevundimonas sp., Microbacterium sp., Sphingomonas sp., and Variovorax sp., the method including the step of initially administering to the animal the effective amount of the composition during the first and second week of the life of the animal.

2. The method of claim 1, wherein the increase in protection is caused by modulation of one or more of the growth-related pathways.

3. The method of claim 1, whereby the composition is mixed with a feed ration portion prior to feeding the animal.

4. The method of claim 1, wherein the composition is fed to the animal in an amount providing from about 0.5 lbs. composition per ton of finished feed to about 11.0 lbs. composition per ton of finished feed.

5. The method of claim 1, wherein the composition is fed to the animal in an amount providing from about 1.0 lbs. composition per ton of finished feed to about 5.0 lbs. composition per ton of finished feed.

6. The method of claim 1, wherein the composition is fed to the animal in an amount providing from about 3.0 lbs. composition per ton of finished feed to about 4.0 lbs. composition per ton of finished feed.

7. The method of claim 1 wherein administration of the composition selectively modulates Toll-like receptors (TLRs) for the prevention and treatment of viral diseases such as avian influenza via a direct effect on innate and adaptive immune pathways.

8. A method for slowing the transmission of avian influenza from an infected bird to a disease-free bird, the method including the steps of administering to the bird an effective amount of a vaccine together with feeding the bird an effective amount of a composition, the composition consisting essentially of a lipopolysaccharide derived from fresh water algal biomass containing Gram-negative bacteria, the composition including a Lipid A fraction of the lipopolysaccharide, the Lipid A fraction being formed through more than one extraction from the Gram-negative bacteria followed by acid hydrolysis, the method including the step of feeding the animal the effective amount of the composition during the first fourteen days of life resulting in the alteration of the immune system consistent with a bolstered innate immune response, whereby the transmission of avian influenza is delayed when compared with such a time interval experienced without treatment.

9. The method of claim 8, wherein the Gram-negative bacteria is selected from the group consisting of Brevundimonas sp., Microbacterium sp., Sphingomonas sp., and Variovorax sp.

10. The method of claim 8, whereby the composition is mixed with a feed ration portion prior to feeding to the animal.

11. The method of claim 8, wherein the composition is fed to the animal in an amount providing from about 0.5 lbs. composition per ton of finished feed to about 11.0 lbs. composition per ton of finished feed.

12. The method of claim 8, wherein the lipopolysaccharide is derived from Gram-negative bacteria present in an algal biomass and wherein the composition is fed to the animal in an amount providing from about 1.0 lbs. composition per ton of finished feed to about 5.0 lbs. composition per ton of finished feed.

13. The method of claim 8, wherein the lipopolysaccharide is derived from Gram-negative bacteria present in an algal biomass and wherein the composition is fed to the animal in an amount providing from about 3.0 lbs. composition per ton of finished feed to about 4.0 lbs. composition per ton of finished feed.

14. The method of claim 8, wherein the alteration of the immune system is caused by modulation of one or more of the growth-related pathways,

15. A method for increasing the level of immunity against avian disease in poultry, the method including the steps of administering to the bird an effective amount of a vaccine together with feeding the bird an effective amount of a composition, the composition being in the form of a lipopolysaccharide derived from a biomass containing Gram-negative bacteria in an amount effective to minimize the risk of the animal becoming infected with avian disease, the lipopolysaccharide being from a biomass and wherein the composition is fed to the animal in an amount providing from about 0.5 lbs. composition per ton of finished feed to about 11.0 lbs. composition per ton of finished feed, the method including the step of feeding the animal an effective amount of the composition during the first fourteen days of life, whereby the immune system is altered consistent with a bolstered innate immune response.

16. The method of claim 15, wherein the Gram-negative bacteria is selected from the group consisting of Brevundimonas sp., Microbacterium sp., Sphingomonas sp., and Variovorax sp.

17. The method of claim 15, wherein the avian disease against which the poultry is immunized against is Infectious Bursal Disease.

18. The method of claim 15, wherein the avian disease against which the poultry is immunized against is avian influenza.

19. The method of claim 15, whereby the composition is mixed with a feed ration portion prior to feeding to the animal.

20. The method of claim 15, wherein biomass is selected from the group consisting of an algal biomass and a bacterial biomass.
